(19) United States
(12) Patent Application Publication
(10) Pub. No.: US 2023/0041417 A1
(43) Pub. Date: Feb. 9, 2023

(54) METHOD OF DETERMINING SKIN AGING

(71) Applicant: Johnson & Johnson Consumer Inc., Skillman, NJ (US)

(72) Inventors: Cecilia Brun, Saint Cyr l'Ecole (FR); Simarna Kaur, Neshanic Station, NJ (US); Thierry Oddos, Clamart (FR)

(21) Appl. No.: 17/878,371

(22) Filed: Aug. 1, 2022

Related U.S. Application Data
(60) Provisional application No. 63/228,959, filed on Aug. 3, 2021.

(51) Int. Cl. C12Q 1/6883; G01N 33/50

(52) U.S. Cl. CPC C12Q 1/6883; G01N 33/5044; G01N 33/5091; C12Q 2600/136; C12Q 2600/158

(57) ABSTRACT

The present invention provides methods of diagnosing and treating fast aging skin, especially in young subjects, by measuring and/or adjusting a property selected from cell proliferation rate, NFKB1 gene expression, CDKN1A gene expression, CDKN2A gene expression, CEBPB gene expression, and combinations thereof.

### CROSS-REFERENCE TO RELATED APPLICATIONS

This application claims the benefit of U.S. provisional application 63/228,959 filed on Aug. 3, 2022, the complete disclosure of which is hereby incorporated herein by reference for all purposes.

### FIELD OF THE INVENTION

The present invention provides methods of diagnosing and treating fast aging skin, especially in young subjects, by measuring and/or adjusting in such skin the cell proliferation rate, NFKB1 gene expression, CDKN1A gene expression, CDKN2A gene expression, or CEBPB gene expression. Methods of screening active agents and other treatments for fast aging skin are also provided.

### BACKGROUND OF THE INVENTION

Skin aging is often characterized by an impairment of dermal fibroblasts function inducing loss of extracellular matrix as well as poor regeneration capacities, ultimately causing wrinkling and sagging. Among several features, loss of proliferation capacities is known to be one of the key markers of fibroblast aging associated with cellular senescence. Although significant differences in cellular proliferation can be evidenced in young populations as compared to older ones, a higher variability is often observed in the younger groups, raising the question of higher inter-donor variability in the young population.

Cellular senescence was originally described by Hayflick and Moorhead in 1961, introducing the theory of cellular aging in which, cells have limited division capacities. L. Hayflick, PS. Moorhead, Exp Cell Res 25 (1961) 585-621. This loss of proliferative potential leads to a cell cycle arrest specific to cellular senescence. It is accompanied by a change in cell morphology, an increased senescence-associated β-galactosidase activity and the release of various cytokines, chemokines, growth factors and proteases, also called the senescence-associated secretory phenotype (SASP).

JAm Acad Dermatol 2018;78:29-39 discusses age-induced and photoinduced changes in gene expression profiles in the skin of Caucasian women aged 20 to 74. Gene expression and ontologic analysis revealed progressive changes from the 20’s to the 70’s in pathways related to oxidative stress, energy metabolism, senescence, and epidermal barrier. The gene expression patterns from a subset of women who were younger-appearing were found to be similar to those in women who were actually younger. In particular, while the authors noted increased CDKN2A gene expression and aged appearance in in sun exposed facial skin, they also stated CDKN2A expression remained relatively unchanged in photo protected buttocks skin samples across the age groups.

Cell Res. 2015 May;25(5):574-87. doi: 10.1038/cr.2015.36 describes the use of three-dimensional human facial morphologies as robust aging markers. In this paper researchers used 3D facial imaging of volunteers to determine facial features of aging, and by comparing their algorithm-predicted age to their chronological age determined slow and fast ager profiles. The results were compared with the blood serum compositions of the volunteers. No analyses on the cellular or genetic level were made, the method only being based on extemal/visual signs of aging. Facial imaging diagnosed fast agers only when visible signs of aging appeared.

Applicants have now discovered that, surprisingly, young skin may in fact be fast aging, containing fast aging cells, despite a youthful clinical appearance. The inventors have identified several biological pathways that signal fast aging skin cells. These may in turn be treated, altered, or regulated to slow the rate of fast-aging skin. This is particularly advantageous because young subjects with fast aging skin may not be able to otherwise determine the need to treat their skin for signs of aging.

### SUMMARY OF THE INVENTION

The present invention provides a method of diagnosing fast aging skin in a young subject, comprising measuring in a skin sample of a subject age 40 years or less cell proliferation rate and expression of a gene selected from the group consisting of NFKB1, CDKN1A, CDKN2A, CEBPB, and combinations thereof.

The present invention also provides a method of evaluating the efficacy of a treatment for fast aging skin, comprising in sequence: (a) applying the treatment to skin of a subject age 40 years or less; and (b) measuring in a skin sample of the subject cell proliferation rate and expression of a gene selected from the group consisting of NFKB1, CDKN1A, CDKN2A, CEBPB, and combinations thereof.

The present invention further provides a method of screening active agents for treatment of fast aging skin, comprising: (a) applying said active agents to skin samples from subjects age 40 years or less; (b) measuring in the skin samples cell proliferation rate and expression of a gene selected from the group consisting of NFKB1, CDKN1A, CDKN2A, CEBPB, and combinations thereof; (c) comparing said measurements with controls; and (d) selecting said active agents that provide at least one of the following compared to the controls: decreased skin fibroblast population doubling time, downregulation of NFKB1 gene expression, downregulation of CDKN1A gene expression, downregulation of CDKN2A gene expression, and downregulation of CEBPB gene expression.

### BRIEF DESCRIPTION OF THE DRAWING

FIG. 1 is a dot plot of time (days) versus mean cell count (x 10,000) at days 0, 4 and 10 for the donors described in Example 1.

FIG. 2 is a volcano plot summarizing the results of the differential analysis on gene expression between high and low proliferative fibroblasts in Example 2.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference.

As used herein, a “product” is optionally in finished packaged form. In one embodiment, the package is a container such as a plastic, metal or glass tube or j ar containing the composition. The product may further contain additional packaging such as a plastic or cardboard box for storing such container. In one embodiment, the product comprises a composition of the invention and contains instructions directing the user to apply the composition to the skin or hair.

As used herein, “topical application” means directly laying on or spreading on outer skin, the scalp, or hair, e.g., by use of the hands or an applicator such as a wipe, roller, or spray.

As used herein, “cosmetically acceptable” means the ingredients the term describes are suitable for use in contact with tissues (e.g., the skin or hair) without undue toxicity, incompatibility, instability, irritation, allergic response, or the like.

As used herein, an “active agent” is a compound (synthetic or natural) that has a cosmetic or therapeutic effect on the skin or hair.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “cosmetically effective amount” means an amount sufficient for treating or preventing one or more signs of skin aging, but low enough to avoid serious side effects. The cosmetically effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

As used herein, “signs or aging” or “signs of skin aging” include the presence of lines including fine lines and wrinkles, loss of elasticity, uneven skin, blotchiness, diminished skin thickness, and abnormal or diminished synthesis of collagen, glycosaminoglycans, proteoglycans, elastin, or glycoproteins including fibronectin. In one embodiment, the sign of aging is selected from the presence of lines, fine lines, wrinkles, loss of elasticity, and abnormal or diminished synthesis of collagen, glycosaminoglycans, proteoglycans, elastin, or glycoproteins including fibronectin.

As used herein, “wrinkle” includes fine lines, fine wrinkles, or coarse wrinkles. Examples of wrinkles include, but are not limited to, fine lines around the eyes (e.g., “crow’s feet”), forehead and cheek wrinkles, frown-lines, and laugh-lines around the mouth.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “improving the firmness of skin” means the enhancing of the firmness or elasticity of the skin, preventing the loss of firmness or elasticity of skin, or preventing or treating sagging, lax and loose skin. The firmness or elasticity of the skin can be measured by use of a cutometer. See Handbook Of Non-Invasive Methods And The Skin, eds. J. Serup, G. Jemec & G. Grove, Chapter 66.1 (2006). The loss of skin elasticity or firmness may be a result of a number of factors, including but not limited to aging, environmental damage, or the result of an application of a cosmetic to the skin.

As used herein, “improving the texture of skin” means the smoothing of the surface of the skin to remove either bumps or crevasses on the skin surface.

As used herein, “improving the appearance of wrinkles in skin” means preventing, retarding, arresting, or reversing the process of wrinkle and fine line formation in skin.

As used herein, “prevention” or “preventing” refers to a reduction of the risk of acquiring a given condition, disease, or disorder.

More broadly, the methods and compositions of the invention may also be used to treat or prevent cosmetic, dermatological, or other conditions and disorders including, but not limited to, infections, deranged or disordered cutaneous or mucocutaneous tissue relevant to skin, nail and hair; oral, vaginal and anal mucosa; disturbed keratinization; inflammation; changes associated with intrinsic and extrinsic aging, and others which may or may not be related to cutaneous system. The manifestations include, but are not limited to, oily skin; acne; rosacea; age spots; blemished skin; blotches; cellulite; dermatoses; dermatitis; skin, nail and hair infections; dandruff; dryness or looseness of skin, nail and hair; xerosis; inflammation, or eczema; elastosis; herpes; hyperkeratosis; hyperpigmented skin; ichthyosis; keratoses; lentigines; melasmas; mottled skin; pseudofolliculitis barbae; photoaging and photodamage; pruritus; psoriasis; skin lines; stretch marks; thinning of skin, nail plate and hair; warts; wrinkles; oral or gum disease; irritated, inflamed, red, unhealthy, damaged or abnormal mucosa, skin, hair, nail, nostril, ear canal, anal or vaginal conditions; breakdown, defective synthesis or repair of dermal components; abnormal or diminished synthesis of collagen, glycosaminoglycans, proteoglycans and elastin, as well as diminished levels of such components in the dermis; uneven skin tone; uneven and rough surface of skin, nail and hair; loss or reduction of skin, nail and hair resiliency, elasticity and recoilability; laxity; lack of skin, nail and hair lubricants and luster; fragility and splitting of nail and hair; yellowing skin; reactive, irritating or telangiectatic skin; and dull and older-looking skin, nail and hair. In addition, the compositions of the current invention can be used for general care of skin, nail and hair; to improve skin texture and pores, flakiness and redness; to make skin soft, smooth, fresh, balanced, visibly clear, even-toned and brighter; to increase skin fullness and plumpness; and for skin bleach and lightening and wound healing; to reduce or prevent sweating or perspiration of underarm, crotch, palm, or other parts of the body.

As used herein, the term “subject” means a human. The subject may be a “young” subject, that is age 40 years or less. The subject may be age 35 years or less. The subject may be age 20-35 years.

As used herein, “fast aging skin” means skin manifesting one or more subclinical signs of aging similar to skin having an older chronological age.

As used herein, “slow aging skin” means skin manifesting one or more subclinical signs of aging constituent with the chronological age of such skin.

As used herein, a “subclinical sign of aging” means phenotypic features such as cellular or molecular signs of aging that are not yet perceived visually.

As used herein, “control” means a reference value for skin having substantially the same, i.e., within about 5 years lesser or greater, chronological age as the skin sample being tested.

Unless otherwise indicated, a percentage or concentration refers to a percentage or concentration by weight (i.e., % (W/W). Unless stated otherwise, all ranges are inclusive of the endpoints, e.g., “from 4 to 9” includes the endpoints 4 and 9.

### METHOD OF DIAGNOSING FAST AGING SKIN

The invention relates a method of diagnosing fast aging skin in a subject.

Preferably, the subject is a young subject.

Preferably, the skin is facial skin. For example, the skin may be from the cheeks, forehead, chin, eye area, or neck, However, skin from any other part of the body may be used, for example the arms, hands, torso, or legs.

Preferably, the skin sample is from the stratum corneum.

The method comprises measuring in a skin sample of a subject a property selected from the group consisting of cell proliferation rate, NFKB1 gene expression, CDKN1A gene expression, CDKN2A gene expression, CEBPB gene expression, and combinations thereof.

The method may comprise measuring in a skin sample cell proliferation rate and expression of a gene selected from the group consisting of NFKB1, CDKN1A, CDKN2A, CEBPB, and combinations thereof.

The method may comprise measuring in a skin sample of a subject age 40 years or less cell proliferation rate and expression of a gene selected from the group consisting of NFKB1, CDKN1A, CDKN2A, CEBPB, and combinations thereof.

The sample may be collected in a variety of ways, as known in the art, including punch biopsies or non-invasive methods such as tape stripping, measuring in-vivo tryptophan fluorescence by using instruments such as SkinSkan or other similar methods, Confocal Raman or Reflectance Confocal Microscopy or other microscope/spectroscopy methods, microneedle patches, or other types of adhesive patch-based skin biopsy devices.

Preferably, the sample is collected by a non-invasive method.

The sample may be analyzed for cell proliferation rate. In such case, skin fibroblast population doubling time, for example in days, of the sample may be measured using methods know in the art. For example, the Proliferation Assay set forth below in Example 1 may be used to measure the cell proliferation rate.

Skin in which the population doubling time is at least 4 days may be diagnosed as fast aging skin.

The sample may be analyzed for one or more of the following gene expressions. Transcriptomic analysis methods are well known in the art. For example, the Transcriptomic Analysis method set forth below in Example 2 may be used.

The sample may be analyzed for NFKB 1 gene expression.

Skin in which the NFKB1 gene expression is upregulated compared with a control may be diagnosed as fast aging skin.

The sample may be analyzed for CDKN1A gene expression.

Skin in which the CDKN1A gene expression is upregulated compared with a control may be diagnosed as fast aging skin.

The sample may be analyzed for CDKN2A gene expression.

Skin in which the CDKN2A gene expression is upregulated compared with a control may be diagnosed as fast aging skin.

The sample may be analyzed for CEBPB gene expression.

Skin in which the CEPBP gene expression is upregulated compared with a control may be diagnosed as fast aging skin.

One of the above genes may be considered upregulated if its expression is at least about 20% greater than the average expression of the same gene in a fibroblast population from donors within about +/- 5 years in age.

One of the above genes may be considered upregulated if its expression is at least about 30% greater than the average expression of the same gene in a fibroblast population from donors within about +/- 5 years in age.

### METHOD OF EVALUATING THE EFFICACY OF A TREATMENT FOR FAST AGING SKIN

The invention also provides a method of evaluating the efficacy of a treatment for fast aging skin.

The subject and skin may be selected and collected as described above. The subject may be age 40 years or less.

The method comprises first applying the treatment to be evaluated to the skin of a subject. Next, one of the following properties may be measured in a sample of the subject’s skin: cell proliferation rate, NFKB1 gene expression, CDKN1A gene expression, CDKN2A gene expression, CEBPB gene expression, and combinations thereof. Cell proliferation along with one or more of NFKB1 gene expression, CDKN1A gene expression, CDKN2A gene expression, and CEBPB gene expression may be measured.

Optionally, prior to applying the treatment to the skin of the subject, one or more of the same properties may be initially measured to provide benchmarks. These may be used as controls.

The samples may be analyzed for cell proliferation rate as described herein. For example, the Proliferation Assay set forth below in Example 1 may be used to measure the cell proliferation rate.

If the treatment provides the skin sample with a population doubling time of less than 4 days it may be deemed efficacious for treating fast aging skin.

The samples may be analyzed for one or more of the following gene expressions. Transcriptomic analysis methods are well known in the art. For example, the Transcriptomic Analysis method set forth below in Example 2 may be used.

The samples may be analyzed for NFKB1 gene expression.

If the treatment provides a downregulation of NFKB1 gene expression it may be deemed efficacious for treating fast aging skin.

The sample may be analyzed for CDKN1A gene expression.

If the treatment provides a downregulation of CDKN1A gene expression it may be deemed efficacious for treating fast aging skin.

The sample may be analyzed for CDKN2A gene expression.

If the treatment provides a downregulation of CDKN2A gene expression it may be deemed efficacious for treating fast aging skin.

The sample may be analyzed for CEBPB gene expression.

If the treatment provides a downregulation of CEBPB gene expression it may be deemed efficacious for treating fast aging skin.

One of the above treatments may be considered efficacious if decreases the expression of the gene being analyzed by at least about 20%.

One of the above treatments may be considered efficacious if decreases the expression of the gene being analyzed by at least about 30%.

### TREATMENTS

Treatments include any means of amelioration, prophylaxis, or reversal of a skin condition, disease, or disorder. They may inhibit, slow the progression, or delay the onset of such skin condition, disease, or disorder, either physically, e.g., stabilization of a discernible symptom, physiologically, e.g., stabilization of a physical parameter, or both.

The treatment may be administered by topical application.

The treatment may be administered orally, i.e., as an ingestible.

The treatment may be topical application of a cosmetic composition.

The composition may contain one or more active agents. The active agents may be cosmetically acceptable active agents. The active agents may be anti-aging agents.

Active agents include for example anti-aging agents, anti-acne agents, shine control agents, anti-microbial agents, anti-inflammatory agents, anti-mycotic agents, anti-parasite agents, external analgesics, sunscreens, photoprotectors, antioxidants, keratolytic agents, surfactants, moisturizers, nutrients, vitamins, energy enhancers, anti-perspiration agents, astringents, deodorants, firming agents, anti-callous agents, and agents for hair and/or skin conditioning

The amount of active agent in the composition may range from about 0.001% to about 20% by weight of the composition, e.g., about 0.005% to about 10% by weight of the composition, such as about 0.01% to about 5% by weight of the composition, based on the total weight of the composition.

The active agent may be selected for instance from alpha hydroxy acids, polyhydroxy acids, dipeptides including N-acyl dipeptide derivatives, tripeptides, benzoyl peroxide, D-panthenol carotenoids, retinoids such as retinol and retinyl palmitate, ceramides, polyunsaturated fatty acids, essential fatty acids, enzymes such as laccase, enzyme inhibitors, minerals, hormones such as estrogens, steroids such as hydrocortisone, 2-dimethylaminoethanol, copper salts such as copper chloride, peptides such as argireline and syn-ake, those containing copper, coenzyme Q10, amino acids such as proline, vitamins, lactobionic acid, acetyl-coenzyme A, niacin, riboflavin, thiamin, ribose, electron transporters such as NADH and FADH2, natural extracts such as those from aloe vera, feverfew, oatmeal, dill, blackberry, princess tree, lemon aspen, resorcinols such as 4-hexyl resorcinol, curcuminoids, sugar amines such as N-acetyl glucosamine, and derivatives and mixtures thereof.

Examples of vitamins include, but are not limited to, vitamin A, vitamin B’s such as vitamin B3, vitamin B5, and vitamin B12, vitamin C, vitamin K, and different forms of vitamin E like alpha, beta, gamma or delta tocopherols or their mixtures, and derivatives thereof.

Examples of other hydroxy acids include, but are not limited, to glycolic acid, lactic acid, malic acid, salicylic acid, citric acid, and tartaric acid.

Examples of antioxidants include, but are not limited to, water-soluble antioxidants such as sulfhydryl compounds and their derivatives (e.g., sodium metabisulfite and N-acyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis.

The cosmetic composition may further include a cosmetically acceptable topical carrier. The carrier may make up from about 25% to about 99.99%, by weight, of the composition (e.g., from about 80% to about 99%, by weight, of the composition). In a preferred embodiment of the invention, the cosmetically acceptable topical carrier includes water.

The compositions may be made into a wide variety of product types that include but are not limited to lotions, creams, gels, sticks, sprays, ointments, cleansing liquid washes and solid bars, shampoos and hair conditioners, hair fixers, pastes, foams, powders, mousses, shaving creams, wipes, patches, hydrogels, film-forming products, facial masks and skin masks, films and make-up such as foundations, and mascaras. These product types may contain a variety of cosmetically acceptable topical carriers including, but not limited to solutions, suspensions, emulsions such as microemulsions and nanoemulsions, gels, solids and liposomes. The following are non-limiting examples of such carriers. Other carriers can be formulated by those of ordinary skill in the art.

The compositions useful in the present invention can be formulated as solutions. Solutions typically include an aqueous or organic solvent (e.g., from about 50% to about 99.99% or from about 90% to about 99% of a cosmetically acceptable aqueous or organic solvent). Examples of suitable organic solvents include propylene glycol, polyethylene glycol, polypropylene glycol, glycerol, 1,2,4-butanetriol, sorbitol esters, 1,2,6-hexanetriol, ethanol, and mixtures thereof.

Compositions useful in the subject invention may be formulated as a solution comprising an emollient. Such compositions preferably contain from about 2% to about 50% of an emollient(s). As used herein, “emollients” refer to materials used for the prevention or relief of dryness, such as by preventing the transepidermal loss of water from the skin. Examples of emollients include, but are not limited to, those set forth in the International Cosmetic Ingredient Dictionary and Handbook, eds. Pepe, Wenninger and McEwen, pp. 2930-36 (The Cosmetic, Toiletry, and Fragrance Assoc., Washington, D.C., 9th Edition, 2002) (hereinafter “ICI Handbook”). Examples of particularly suitable emollients include vegetable oils, mineral oils, fatty esters, and the like.

A lotion can be made from such a solution. Lotions typically contain from about 1% to about 20% (e.g., from about 5% to about 10%) of an emollient(s) and from about 25% to about 90% (e.g., from about 60% to about 80%) of water.

Another type of product that may be formulated from a solution is a cream. A cream typically contains from about 5% to about 50% (e.g., from about 10% to about 20%) of an emollient(s) and from about 25% to about 85% (e.g., from about 50% to about 75%) of water.

The composition of the present invention may include water or alternatively be anhydrous or be an ointment that includes no water but organic and/or silicone solvents, oils, lipids and waxes. An ointment may contain a simple base of animal or vegetable oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook pp. 2979-84.

The composition may be formulated as an emulsion. If the topical carrier is an emulsion, from about 1% to about 10% (e.g., from about 2% to about 5%) of the topical carrier contains an emulsifier(s). Emulsifiers may be nonionic, anionic or cationic. Examples of emulsifiers include, but are not limited to, those set forth in the ICI Handbook, pp.2962-71.

Lotions and creams can be formulated as emulsions. Typically such lotions contain from 0.5% to about 5% of an emulsifier(s). Such creams typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contain between about 0.1% and 5%, by weight, of such gelling agents.

The compositions can also be formulated into a solid formulation (e.g., a wax-based stick, soap bar composition, powder, or a wipe containing powder).

The compositions may contain, in addition to the above components, a wide variety of additional oil-soluble materials and/or water-soluble materials conventionally used in compositions for use on skin and hair, at their art-established levels.

Various other materials may also be present in the composition, as known in the art. These include humectants, pH adjusters, chelating agents (e.g., EDTA), fragrances, dyes, and preservatives (e.g., parabens).

The compositions and products containing such compositions may be prepared using methodology that is well known in the art.

In one embodiment, the composition has a low pH. For example, the pH may be less than about 4 or less than about 3.3. The composition is not required to have a low pH, however.

The topical composition may comprise a buffering agent such as lactic acid, citric acid, malic acid, tartaric acid, gluconic acid, or gluconolactone. Preferably the buffering agent is lactic acid.

Typically, the composition contains about 3 to about 12, or about 4 to about 8, weight percent of buffering agent.

### METHOD OF SCREENING ACTIVE AGENTS FOR TREATMENT OF FAST AGING SKIN

The invention provides a method of screening active agents for treatment of fast aging skin as follows.

The subject and skin may be selected and collected as described above.

The active agents to be screened include the ones set forth above. For example, the active agents to be screened may be anti-aging actives.

The method comprises: (a) applying the active agents to the skin samples; (b) measuring in said skin samples a property selected from cell proliferation rate, NFKB1 gene expression, CDKN1A gene expression, CDKN2A gene expression, CEBPB gene expression and combinations thereof; (c) comparing said property with a control; and (d) selecting said active agents that provide at least one of the following compared to the control: decreased skin fibroblast population doubling time, downregulation of NFKB1 gene expression, downregulation of CDKN1A gene expression, downregulation of CDKN2A gene expression, and downregulation of CEBPB gene expression.

The method may comprise : (a) applying said active agents to skin samples from subjects age 40 years or less; (b) measuring in the skin samples cell proliferation rate and expression of a gene selected from the group consisting of NFKB1, CDKN1A, CDKN2A, CEBPB, and combinations thereof; (c) comparing said measurements with controls; and (d) selecting said active agents that provide at least one of the following compared to the controls: decreased skin fibroblast population doubling time, downregulation of NFKB1 gene expression, downregulation of CDKN1A gene expression, downregulation of CDKN2A gene expression, and downregulation of CEBPB gene expression.

The samples may be analyzed for cell proliferation rate as described herein. For example, the Proliferation Assay set forth below in Example 1 may be used to measure the cell proliferation rate.

The samples may be analyzed for the gene expressions using transcriptomic analysis methods, which are well known in the art. For example, the Transcriptomic Analysis method set forth below in Example 2 may be used.

### Example 1

The proliferation rates of skin cells from young and old donors were tested in the following study. The results were compared with expert clinical grading of the donors by dermatologists.

Volunteers were divided into two groups:
Group 1: “Young Donors” age range 20-33 years (n=9), and
Group 2: “Old Donors” age range 61-68 years (n=9).

Cell proliferation rates were analyzed as follows. Dermal fibroblasts were isolated from outgrowth of two 3 mm-punch biopsies taken on the volar forearm and were cultured in Dulbecco’s modified Eagle’s medium supplemented with 10% fetal bovine serum, 1% fungizone, 0.04% de Gentamycin and 1% Glutamax in a 90% humidified incubator with 5% CO2 at 37° C. Cells were amplified and weekly passaged.

Fibroblasts from both groups were assessed for their proliferation rates using the following Proliferation Assay.

### Proliferation Assay

Cells were plated in a 24-well plate at 4.10^4 cell/well and incubated for 4 or 10 days in Dulbecco’s Modified Eagle’s Medium supplemented with 10% fetal bovine serum, 1% fungizone, 0.04% de Gentamycin and 1% Glutamax in a 90% humidified incubator with 5% CO₂ at 37° C. On day 4 or day 10, cells were detached and counted with a Coulter Z1ST (Beckman Coulter France) cell counter.

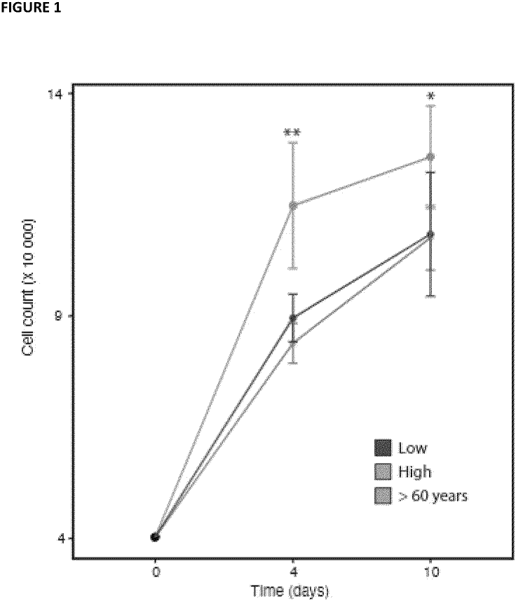

FIG. 1 shows the proliferation rates of the young donors and old donors. It is a dot plot of time (days) versus mean cell count (x 10,000) at days 0, 4 and 10 for the donors. The data shows that, surprisingly, while some young donors display high proliferative capacities consistent with their age, others show low proliferative capacities similar to the one observed in old donors.

Specifically, Table 1 shows the mean population doubling time (“PD Time” in days) for each of the high proliferators and low proliferators within the young donors and old donors at Day 4. Cell proliferation reached a plateau at Day 10. (Because cells should be in the proliferative/exponential phase to calculate the population doubling time, proliferation data from Day 10 was not included in the calculation.)

In Table 1:

PD=[ln(Number of cells collected)-ln(Number of cells seeded)]/ln(2)

PD time=[numbers of days between seeding and collection]/PD.

Young donor, high proliferators had a mean PD Time of 2.76 days. Old donors had a mean PD Time of 4.43 days Young donor, low proliferators unexpectedly had a mean PD Time of 5.44 days, which was not statistically significantly different from the PD Time of the old donors. Table 2 shows the results of the statistical analysis. Statistical analysis was performed using the two-sided Student’s t-Test.

Table 1

| Donor | | PD Time (Days) |
|---|---|---|
| Young High Proliferator | Mean | 2.76 |
| | STD | 0.45 |
| Young Low Proliferator | Mean | 5.44 |
| | STD | 1.00 |
| Old | Mean | 4.43 |
| | STD | 0.62 |

Table 2

| | | |
|---|---|---|
| High Proliferator vs Low Proliferator | High Proliferator vs Old Donor | Low Proliferator vs Old Donor |
| 0.002 | 0.001 | 0.09 |
| *** | *** | NS |

***Statistically Significant
NS Not Statistically Significant

The same donors were expertly graded by a dermatologist for 17 signs of skin aging. Between the young donors and the old donors, 13 of the 17 signs were statistically significantly different (also using the two-sided Student’s t-Test). However, only 3 of the 17 signs were significantly different between the young donor, high proliferators and young donor, low proliferators: dryness, number of frontal wrinkles and depth of frontal wrinkles.

Surprisingly, certain young donors, whose skin appeared young with few clinical signs of aging, possessed a degree of low proliferating skin cells consistent with skin from old donors. This indicates the presence of subclinical aging in young skin and suggests such young skin is fast aging relative to chronological age.

### Example 2

A transcriptomic analysis was performed on fibroblasts from the young donors of Example 1 using the following Transcriptomic Analysis.

### Transcriptomic Analysis

Cells from the young donor group were harvested at 80-90% confluence, pelleted and frozen. Total RNA was extracted using the RNeasy Plus Mini Kit (Qiagen, France) with a Qiacube according to the manufacturer’s instructions. Total RNAs were subjected to transcriptomic microarray profiling.

For transcriptome array data, a background correction followed with a quantile normalization (QN) was performed using the RMA framework as implemented in the limma R package. This procedure is aimed at normalizing the median expression between arrays. Quality-check was performed to properly evaluate the impact of the QN and filtering using extensive data visualization before and after normalization. The differential analysis was conducted using limma to identify genes differentially expressed between high proliferators, i.e., slow agers, and low proliferators, i.e., fast agers. Gene Set Enrichment Analysis (GSEA) was then performed using thefgsea R package to identify sets of genes that are over-represented among genes up- or downregulated using the Molecular Signatures Database (https://www.gsea-msigdb.org/gsea/msigdb) curated genesets.

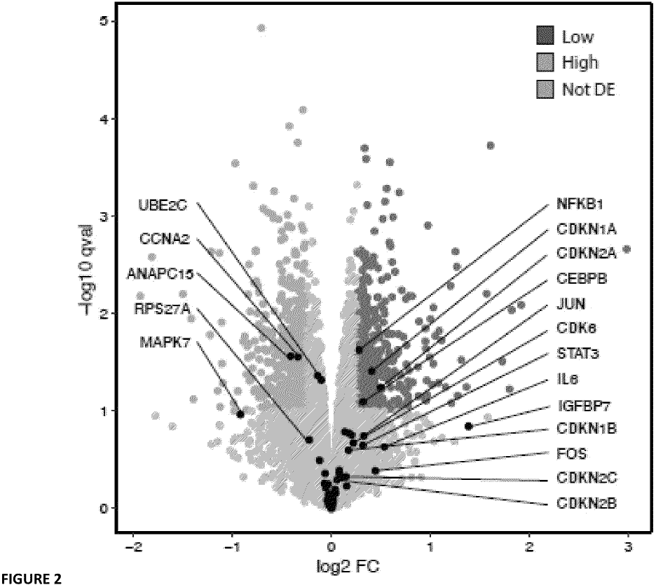

The results are shown in FIG. 2, which is a volcano plot summarizing the results of the differential analysis on gene expression between high and low proliferative fibroblasts. Blue dots depict genes up-regulated in the low proliferative fibroblasts, while orange dots depict up-regulated genes in high proliferative fibroblasts (FDR q < 0.1 & absolute log2 FC > log2(1.2)). The dots highlighted in black correspond to genes belonging to the senescence pathways from the Reactome database.

Genes related to NFKB1, CDKN1A, CDKN2A, and CEBPB were upregulated in the fibroblasts of the young donor low proliferators. These markers are known to be associated with cellular senescence.

These results confirm the existence of a subset of young donors surprisingly exhibiting proliferation capacities similar to those observed in old donors. The low proliferative young fibroblasts displayed features of early senescence. Senescence creates a pro-inflammatory micro-environment known to contribute not only to the appearance of skin aging signs like wound healing defects, wrinkling or sagging, but also to the development of age-related diseases such as cancers.

This data shows that a young population unexpectedly contains both slow skin agers and fast skin agers.

We claim:

1. A method of diagnosing fast aging skin in a young subject, comprising measuring in a skin sample of a subject age 40 years or less cell proliferation rate and expression of a gene selected from the group consisting of NFKB1, CDKN1A, CDKN2A, CEBPB, and combinations thereof.

2. The method of claim 1, further comprising comparing the cell proliferation rate or expression of a gene with a control.

3. The method of claim 2, wherein the control for cell proliferation rate is a skin fibroblast population doubling time of at least 4 days.

4. The method of claim 2, wherein the control for expression of a gene is an expression at least about 20% greater than an average expression of said gene in a fibroblast population from donors within about +/- 5 years in age.

5. The method of claim 1, wherein said subject is age 20 to 35 years.

6. The method of claim 1, wherein the skin sample is obtained by a noninvasive method selected from the group consisting of tape stripping, measuring in-vivo tryptophan fluorescence, microscope/spectroscopy methods, microneedle patches, and adhesive patch-based skin biopsy devices.

7. A method of evaluating the efficacy of a treatment for fast aging skin, comprising in sequence: (a) applying the treatment to skin of a subject age 40 years or less; and (b) measuring in a skin sample of the subject cell proliferation rate and expression of a gene selected from the group consisting of NFKB1, CDKN1A, CDKN2A, CEBPB, and combinations thereof.

8. The method of claim 7, further comprising comparing said measurement with a control.

9. The method of claim 7, wherein said treatment is a topical application of a cosmetic composition.

10. The method of claim 7, wherein the subject is age 20 to 35 years.

11. A method of screening active agents for treatment of fast aging skin, comprising: (a) applying said active agents to skin samples from subjects age 40 years or less; (b) measuring in the skin samples cell proliferation rate and expression of a gene selected from the group consisting of NFKB1, CDKN1A, CDKN2A, CEBPB, and combinations thereof; (c) comparing said measurements with controls; and (d) selecting said active agents that provide at least one of the following compared to the controls: decreased skin fibroblast population doubling time, downregulation of NFKB1 gene expression, downregulation of CDKN1A gene expression, downregulation of CDKN2A gene expression, and downregulation of CEBPB gene expression.
